(19) United States
(12) Patent Application Publication
(10) Pub. No.: US 2024/0122491 A1
(43) Pub. Date: Apr. 18, 2024

(54) AUSCULTATORY SPHYGMOMANOMETER SYSTEM AND METHOD OF USING THE SAME TO MEASURE BLOOD PRESSURE

(71) Applicant: National Taipei University of Technology, Taipei (TW)

(72) Inventors: Ren-Guey LEE, Taipei (TW); Li-Luo HO, Taipei (TW)

(73) Assignee: National Taipei University of Technology, Taipei (TW)

(21) Appl. No.: 17/968,342

(22) Filed: Oct. 18, 2022

(51) Int. Cl. A61B 5/022; A61B 5/00; A61B 7/04

(52) U.S. Cl. CPC A61B 5/02208; A61B 5/02225; A61B 5/02233; A61B 5/725; A61B 7/045; A61B 2562/0247; A61B 2562/028

(57) ABSTRACT

An auscultatory sphygmomanometer system includes a pressure sensing and working module and a cuff. The cuff has a tube connected with the pressure sensing and working module, a gasbag connected with the tube and inflated or deflated according to the working state of the pressure sensing and working module, an audio pickup probe disposed on the gasbag and using a lateral surface of the gasbag as an oscillation membrane, and a cuff body accommodating the gasbag. While the cuff body is wound around a testee's arm, the gasbag is positioned into a first surface neighboring the testee's arm and a second surface opposite thereto and far away from the testee's arm. The connection position where the tube is joined to the gasbag is on the second surface. The sounds picked up by the audio pickup probe are sued to determine the systolic pressure and the diastolic pressure.

### BACKGROUND OF THE INVENTION

### Field of the Invention

The present invention relates an auscultatory sphygmomanometer system and a method of using the same to measure blood pressure, particularly to an auscultatory sphygmomanometer system using the sound signals picked up by the audio pickup probe to determine blood pressure and a method of using the same to measure blood pressure.

### Description of the Prior Art

At present, the frequently-seen sphygmomanometers include the auscultatory sphygmomanometers and the oscillometric sphygmomanometers. The auscultatory sphygmomanometer includes a mercury manometer, a gas pump, a cuff, and a stethoscope. In application, the cuff is wound around the upper arm of a testee; the gas pump inflates a gasbag inside the cuff until the gas pressure inside the gasbag is greater than the heart contracting pressure, whereby the blood flow of the artery is blocked; then, the gasbag is deflated gradually to resume the blood flow of the artery. The heart pulses impact the arterial wall and generate Korotkoff sounds. While the operator hears the Korotkoff sound from the stethoscope for the first time, the pressure value on the mercury manometer is the systolic pressure. While the operator hears the Korotkoff sound for the last time from the stethoscope, the pressure value on the mercury manometer is the diastolic pressure. While the operator is listening to the Korotkoff sounds through the stethoscope, he may also hear some noises, such as the sounds generated by inflation and deflation of the gasbag, and the sounds generated by the rubs between the clothes and the cuff, which make the unprofessional persons hard to determine whether the Korotkoff sound is appearing. Therefore, the operation of the conventional auscultatory sphygmomanometer must rely on professional personnel. Besides, the placement and sensitivity of the stethoscope influences the accuracy of the conventional auscultatory sphygmomanometer.

The oscillometric sphygmomanometer needn't be operated by professional personnel. Therefore, the oscillometric sphygmomanometer is popular in the market. In the conventional oscillometric sphygmomanometer, the systolic pressure is determined by using the average amplitude as the center to find forwards the 50% of the largest amplitude as the systolic pressure, and using the largest amplitude as the center to find backwards the 80% of the largest amplitude as the diastolic pressure. In other words, the conventional oscillometric sphygmomanometer cannot acquire the pressure values directly but must use an estimation method to learn the systolic pressure and the diastolic pressure. Thus, the conventional oscillometric sphygmomanometers of different manufacturers or different designs may output different pressure values.

### SUMMARY OF THE INVENTION

The primary objective of the present invention is to solve the problem that the blood pressure values measured by the conventional oscillometric sphygmomanometer are likely to have errors.

Another objective of the present invention is to solve the problem that unprofessional persons are hard to accurately determine the Korotkoff sounds of the conventional auscultatory sphygmomanometer.

In order to achieve the abovementioned objectives, the present invention provides an auscultatory sphygmomanometer system, which comprises a pressure sensing and working module and a cuff connected with the pressure sensing and working module. The cuff includes a tube connected with the pressure sensing and working module, a gasbag connected with the tube and inflated or deflated according to the working state of the pressure sensing and working module, an audio pickup probe disposed on the gasbag and using a lateral surface of the gasbag as an oscillation membrane, and a cuff body accommodating the gasbag. While the cuff body surrounds the arm of a testee, the gasbag is divided into a first surface neighboring the arm of the testee and a second surface opposite to the first surface and far away from the arm of the testee. The connection position where the tube is joined to the gasbag is on the second surface. The sounds picked up by the audio pickup probe are used to determine the systolic pressure and the diastolic pressure.

In one embodiment, the audio pickup probe has a cone-shaped stethoscope disposed on the surface of the gasbag, and an audio sensor disposed on one end of the cone-shaped stethoscope, which has a smaller diameter.

In one embodiment, the audio sensor is a microelectromechanical system (MEMS) microphone. The response frequency of the MEMS microphone ranges from 10 Hz to 20 kHz. The sensitivity of the MEMS microphone is at least −45 dBV while the sound level calibrator outputs a sound signal having a frequency of 1 kHz and a sound pressure level of 94 dB.

In one embodiment, the pressure sensing and working module includes a gas valve connected with the tube, a gas pump connected with the tube, and a pressure sensor detecting the pressure inside the gasbag.

In one embodiment, the auscultatory sphygmomanometer system includes a signal processor connected with the pressure sensing and working module and the audio pickup probe. The signal processor includes at least one first signal amplifier; a first multiple-band bandpass filter performing signal processing of the sound signals provided by the audio pickup probe; a second-order low pass filter performing signal processing of the pressure signals provided by the pressure sensor to provide pressure values in a direct-current mode; and a second-order bandpass filter performing signal processing of the pressure signals provided by the pressure sensor to provide oscillometric pressure values in an alternating-current mode.

In one embodiment, the first multiple-band bandpass filter includes a first-order RC low pass filter, a second-order Sallen-key low pass filter, a first-order RC high pass filter, and a second-order Sallen-key high pass filter.

In one embodiment, the audio pickup probe includes a front-end signal processor connected with the audio sensor. The front-end signal processor includes a second signal amplifier, and a second multiple-band band pass filter succeeding to the second signal amplifier.

In one embodiment, the second multiple-band band pass filter includes a second-order Chebyshev low pass filter, and a second-order Chebyshev high pass filter.

The present invention also provides a method of using an auscultatory sphygmomanometer system to measure blood pressure, which comprises
Step 1: providing an auscultatory sphygmomanometer system provided by any one of claims 1-8; and
Step 2: inflating the gasbag inflate to a preset value, and then deflating the gasbag under the action of the pressure sensing and working module; the audio pickup probe using the second surface of the gasbag as an oscillation membrane to generate a sound signal; acquiring a systolic pressure while the first Korotkoff sound appears, and acquiring a diastolic pressure while the last Korotkoff sound appears.

In comparison with the conventional technologies, the auscultatory sphygmomanometer system of the present invention is characterized in combining the advantage of the conventional oscillometric sphygmomanometer that needn't be operated by professional personnel and the advantage of the conventional auscultatory sphygmomanometer that can measure blood pressure accurately, wherein the audio pickup probe on the cuff uses one surface of the gasbag as the oscillation membrane, and wherein the sound signals picked up by the audio pickup probe are directly used to determine the systolic pressure and the diastolic pressure.

### BRIEF DESCRIPTION OF THE DRAWINGS

FIG. 1 is a diagram schematically showing an auscultatory sphygmomanometer system according to one embodiment of the present invention.

FIG. 2 is a diagram schematically showing a structure of a cuff according to one embodiment of the present invention.

FIG. 3 is a sectional view schematically showing a cuff according to one embodiment of the present invention.

FIG. 4 is a flowchart of a method of using an auscultatory sphygmomanometer system to measure blood pressure according to one embodiment of the present invention.

FIG. 5 is a diagram showing sound signals and pressures inside a gasbag according to one embodiment of the present invention.

FIG. 6 is a diagram schematically showing the units of an auscultatory sphygmomanometer system according to one embodiment of the present invention.

FIG. 7 is a diagram schematically showing the units of an auscultatory sphygmomanometer system according to another embodiment of the present invention.

FIG. 8 is a diagram schematically showing the units of an auscultatory sphygmomanometer system according to still another embodiment of the present invention.

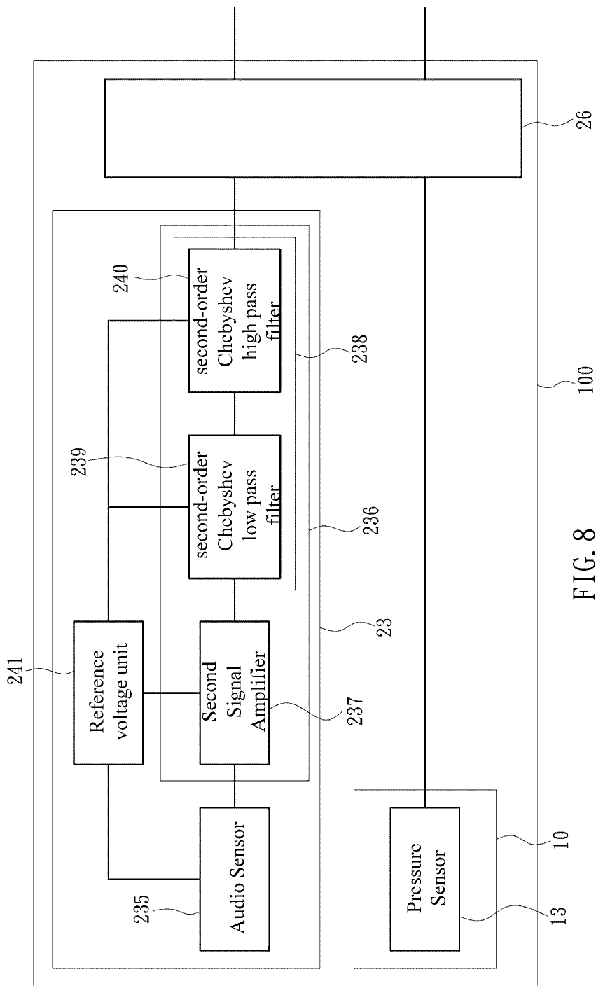

### DETAILED DESCRIPTION OF THE INVENTION

The technical contents of the present invention will be described in detail in cooperation with drawings below.

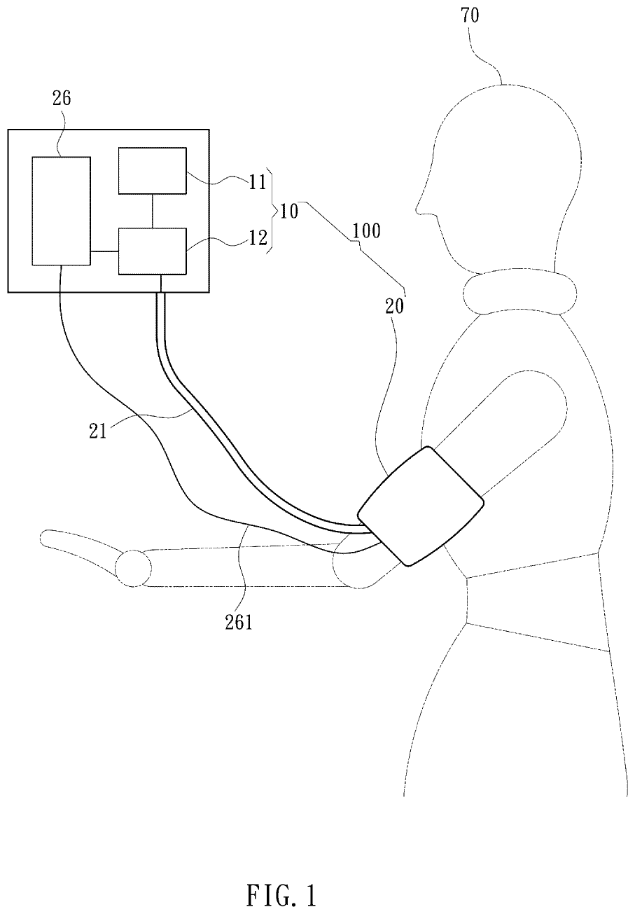

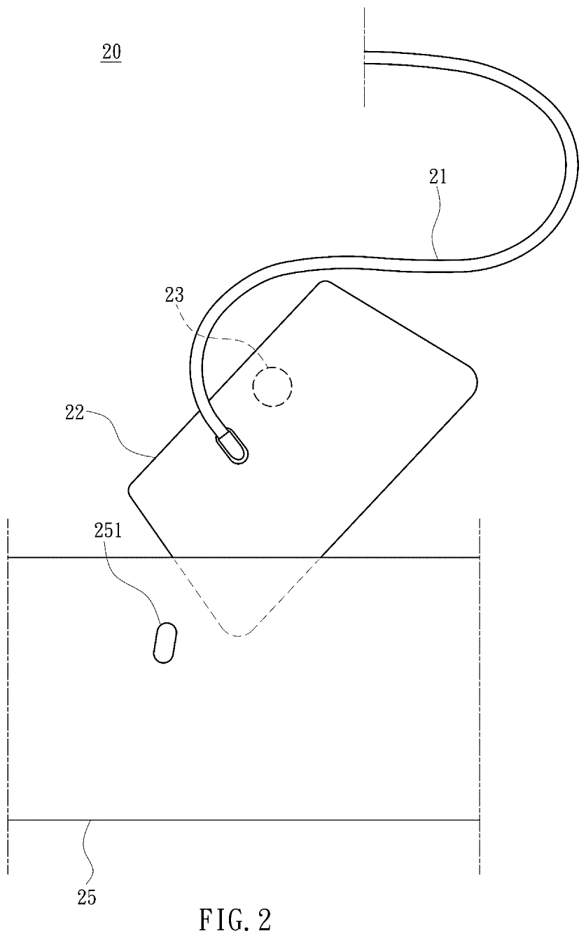

Refer to FIGS. 1-3, FIG. 5 and FIG. 6. The present invention provides an auscultatory sphygmomanometer system 100, which comprises a pressure sensing and working module 10 and a cuff 20 connected with the pressure sensing and working module 10. The pressure sensing and working module 10 includes a gas pump 11, a gas valve 12, and a pressure sensor 13. The gas pump 11 is connected with the cuff 20 and determines whether to inflate the cuff 20 according to the working state of the pressure sensing and working module 10. The gas valve 12 is disposed between the gas pump 11 and the cuff 20 and connected with both to form a gas channel. The gas valve 12 controls the flow direction of the gas and determines whether to inflate or deflate the cuff 20. The pressure sensor 13 is disposed on the gas channel of the cuff 20 and used to measure the gas pressure inside the cuff 20.

The cuff 20 includes a tube 21 connected with the pressure sensing and working module 10, a gasbag 22 connected with the tube 21, an audio pickup probe 23 disposed in the gasbag 22, and a cuff body 25 accommodating the gasbag 22. The tube 21 is connected with the gas valve 12 and the gas pump 11, wherein the gas pump 11 and the gas valve 12 supply gas to the gasbag 22 through the tube 21; the gas inside the gasbag 22 may be exhausted through the tube 21. The volume of the gasbag 22 varies during inflation and deflation. The gasbag 22 interconnects with the pressure sensor 13, and the pressure sensor 13 detects the pressure inside the gasbag 22. The audio pickup probe 23 is disposed on a lateral surface of the gasbag 22 and uses the surface as the oscillation membrane. The gasbag 22 generates oscillations during inflation and deflation. The audio pickup probe 23 acquires a sound signal from the oscillations of the gasbag 22. The cuff body 25 surrounds the arm of a testee 70 during test, and the arm may be a left aim or a right aim of the testee 70. The gasbag 22 may be partitioned into a first surface 221 neighboring the arm of the testee 70, and a second surface 222 opposite to the first surface 221 and far away from the arm of the testee 70. The connection position where the tube 21 is joined to the gasbag 22 is on the second surface 222. One end of the tube 21, which is not connected with the gasbag 22, may penetrate from one opening of the cuff body 25, which is on the second surface 222, lest the inflated gasbag 22 compress the tube 21. The audio pickup probe 23 is also on the second surface 222, whereby to decrease the compression of the inflated gasbag 22 on the audio pickup probe 23 and reduce the influence of the inflated gasbag 22 on sound signals 231.

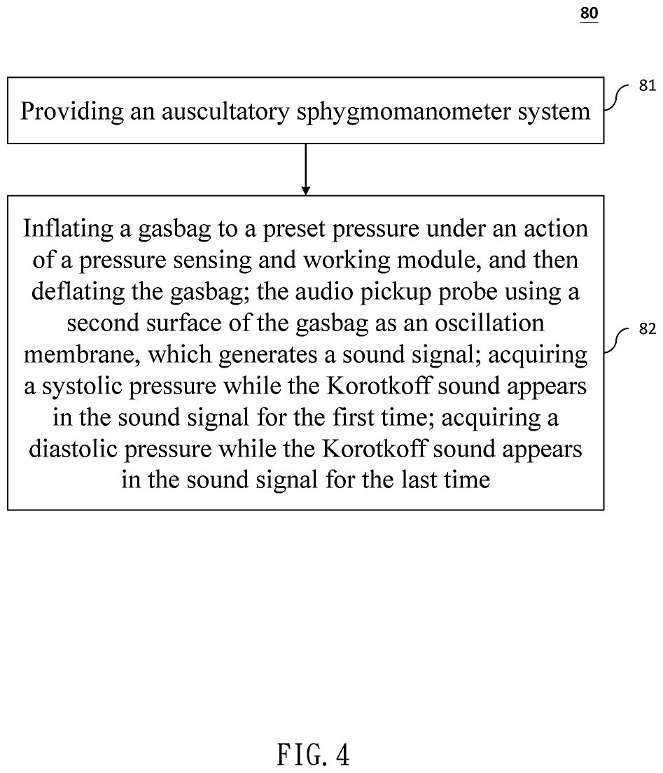

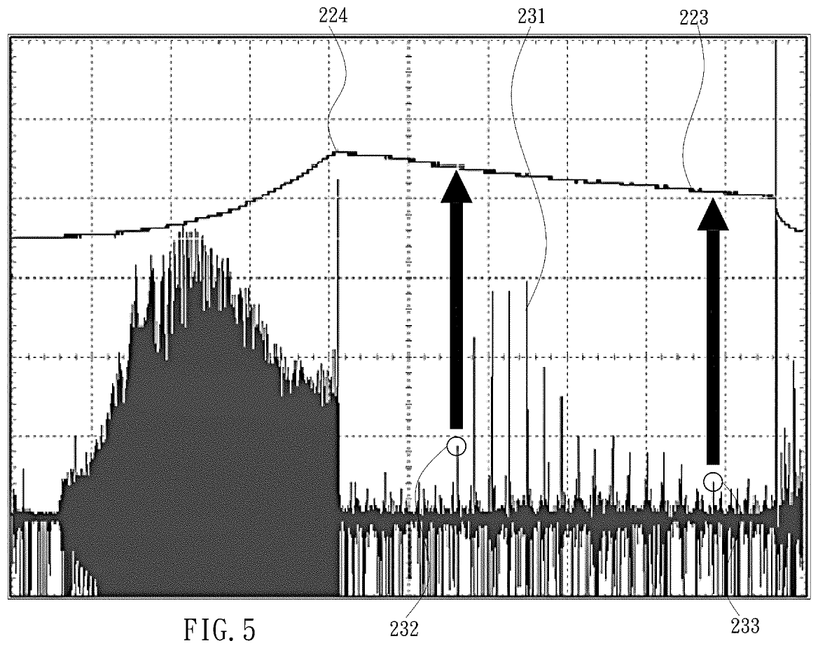

Refer to FIGS. 1-6. The method 80 of measuring blood pressure with the auscultatory sphygmomanometer system 100 is described below. Before the description of the method, FIG. 5 is explained beforehand. FIG. 5 shows the relationship of the pressure curve 223 of the gasbag 22 and the amplitudes of the sound signals 231 of the audio pickup probe 23. In Step 81, provide the auscultatory sphygmomanometer system 100, and wind the cuff 20 around the upper arm of the testee 70. Next, in Step 82, inflate the gasbag 22 under the action of the pressure sensing and working module 10, and the pressure curve 223 of the gasbag 22 rises upward at the same time; while the gasbag 22 is inflated to a preset pressure 224, the pressure sensing and working module 10 stops inflating the gasbag 22. The preset pressure 224 must be greater than the systolic pressure of the testee 70 so that the gasbag 22 can interrupt the blood flow of the testee 70. In the present invention, the preset pressure 224 is 175 mmHg. Next, deflate the gasbag 22 slowly. While the gasbag 22 is deflated to an extent that the blood flow of the testee 70 resumes, the Korotkoff sound appears in the sound signals 231 for the first time, such as the peak 232 in FIG. 5. Thus, the auscultatory sphygmomanometer system 100 takes the pressure of the gasbag 22, which is detected by the pressure sensing and working module 10 at the moment, as the systolic pressure. At the same time, the gasbag 22 is deflated continuously. While the last Korotkoff sound, such as the peak 233 in FIG. 5, appears in the sound signals 231, the auscultatory sphygmomanometer system 100 takes the pressure of the gasbag 22, which is detected by the pressure sensing and working module 10 at the moment, as the diastolic pressure.

It is learned from the above description: the present invention uses the surface of the gasbag 22 as the oscillation membrane, whereby the audio pickup probe 23 can directly acquire the sound signals 231 from the oscillations of the gasbag 22. Therefore, the present invention can exempt the operation of measuring blood pressure from relying on professional personnel. Besides, while the audio pickup probe 23 of the present invention receives the Korotkoff sound, the pressure of the gasbag 22, which is detected at the moment, is taken as the blood pressure. Thus, the present invention is exempted from the problem of inaccurate measurement results of the conventional oscillometric sphygmomanometer wherein blood pressure is obtained via estimation.

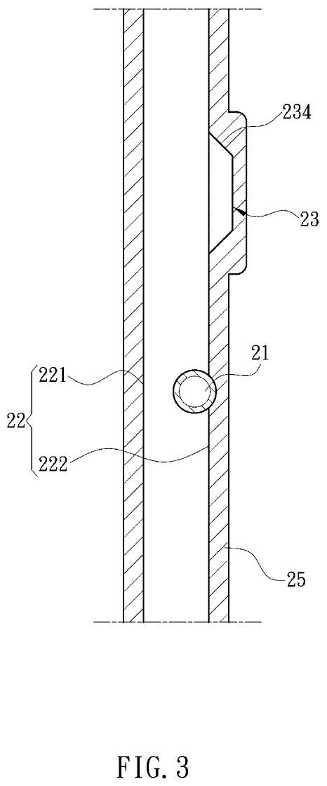

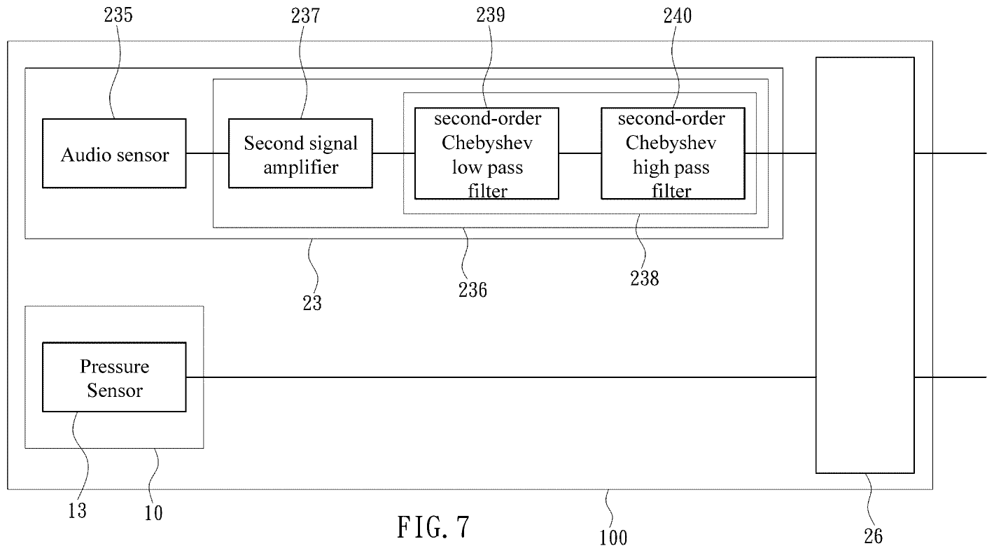

Refer to FIG. 3 and FIG. 7. The audio pickup probe 23 has a cone-shaped stethoscope 234 disposed on the second surface 222 of the gasbag 22, and an audio sensor 235 disposed on one end of the cone-shaped stethoscope 234, which has a smaller diameter. Another end of the cone-shaped stethoscope 234, which has a larger diameter, is connected with the gasbag 22. As mentioned above, the end of the cone-shaped stethoscope 234, which has a smaller diameter, provides a place to dispose the audio sensor 235. The diameter of the cone-shape stethoscope 234 decreases gradually, whereby to amplify the sounds generated by the oscillations of the gasbag 22. In one embodiment, the audio sensor 235 is a microelectromechanical system (MEMS) microphone. The response frequency of the MEMS microphone ranges from 10 Hz to 20 kHz. The sensitivity of the MEMS microphone is at least −45 dBV while the sound level calibrator outputs a sound signal having a frequency of 1 kHz and a sound pressure level of 94 dB.

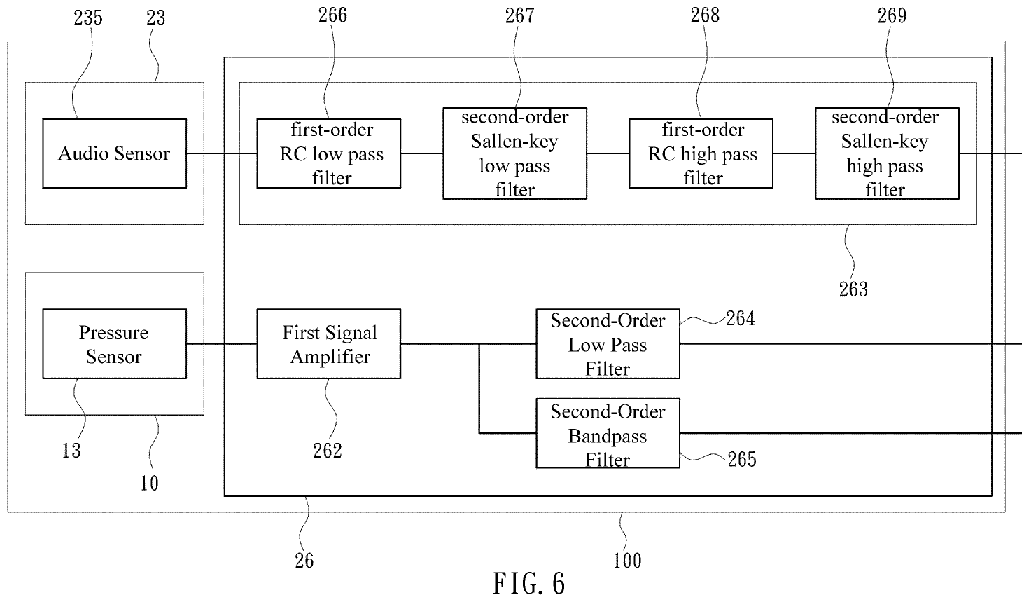

Refer to FIG. 1, FIG. 5, FIG. 6, and FIG. 7. In one embodiment, the auscultatory sphygmomanometer system 100 includes a signal processor 26 connected with the pressure sensing and working module 10 and the audio pickup probe 23. The signal processor 26 is connected with the audio pickup probe 23 through a cable 261. The signal processor 25 includes at least one first signal amplifier 262, a first multiple-band bandpass filter 263, a second-order low pass filter 264; and a second-order bandpass filter 265. The first signal amplifier 262 is connected with the pressure sensor 13, amplifying the voltage by at least +12 dB. If the signal processor 25 includes a plurality of first signal amplifiers 262, the first signal amplifiers 262 are respectively disposed at the front ends of the second-order low pass filter 264 and the second-order bandpass filter 265. The first multiple-band bandpass filter 263 is a circuit including resistors, inductors, and capacitors. The first multiple-band bandpass filter 263 performs signal processing of the sound signals 231 provided by the audio pickup probe 23. In one embodiment, the first multiple-band bandpass filter 263 is a six-band bandpass filter 263, which is set to have a gain of +12 dB, a central frequency of 15 Hz, a −3 dB cutoff frequency of 200 Hz, and a −40 dB cutoff frequency of 500 Hz. The first multiple-band bandpass filter 263 includes a first-order RC low pass filter 266, a second-order Sallen-key low pass filter 267, a first-order RC high pass filter 268, and a second-order Sallen-key high pass filter 269. The second-order low pass filter 264 performs signal processing of the pressure signals provided by the pressure sensor 13 to provide pressure values in a direct-current mode. The second-order bandpass filter 265 performs signal processing of the pressure signals provided by the pressure sensor 13 to provide oscillometric pressure values in an alternating-current mode.

In one embodiment, in order to reduce the influence of the cable 261 on the signal processor 26, the audio pickup probe 23 includes a front-end signal processor 236 connected with the audio sensor 235 and disposed at the front stage of the signal processor 26. The front-end signal processor 236 receives the sound signals 231 from the audio sensor 235 and performs preliminary signal processing of the sound signals 231. The front-end signal processor 236 includes a second signal amplifier 237 and a second multiple-band bandpass filter 238 succeeding to the second signal amplifier 237. The second signal amplifier 237 amplifies the sound signals 231 by at least 20 dB to make the difference of the crest value and the trough value of the Korotkoff sound at least 200 mV. While the sound signals 231 are amplified, the ambient sounds, the sound of the inflation of the gasbag 22, and the sounds of moving the arm of the testee 70 are also amplified simultaneously. The present invention uses the second multiple-band bandpass filter 238 to perform a filtering treatment of the amplified sound signals 231 to filter out the sounds having frequencies of more than 200 Hz, whereby to eliminate the noises, such as the ambient sounds, the sound of the inflation of the gasbag 22, and the sounds of moving the arm of the testee 70. In one embodiment, the second multiple-band bandpass filter 238 is a four-band bandpass filter. The four-band bandpass filter is set to have a gain of 0 dB, a central frequency of 15 Hz, a −3 dB cutoff frequency of 200 Hz, and a −40 dB cutoff frequency of 2000 Hz. The second multiple-band band pass filter 238 includes a second-order Chebyshev low pass filter 239, and a second-order Chebyshev high pass filter 240. In one embodiment, considering the second signal amplifier 237 and the second multiple-band band pass filter 238 jointly use a single power source, the audio pickup probe 23 further includes a reference voltage unit 241, which is connected with the second signal amplifier 237 and the second multiple-band band pass filter 238. The reference voltage unit 241 performs voltage division for the received working voltage and takes ½ working voltage as the reference voltage of the second signal amplifier 237 and the second multiple-band band pass filter 238.

The embodiments described above are only to exemplify the present invention but not to limit the scope of the present invention. Any equivalent modification or variation made according to the shape, structure, characteristics or spirit disclosed by the present invention is to be also included by the scope of the present invention.

What is claimed is:

1. An auscultatory sphygmomanometer system, comprising
a pressure sensing and working module; and
a cuff connected with the pressure sensing and working module and including a tube connected with the pressure sensing and working module, a gasbag connected with the tube and inflated or deflated according to a working state of the pressure sensing and working module, an audio pickup probe disposed on the gasbag and using a lateral surface of the gasbag as an oscillation membrane, and a cuff body accommodating the gasbag,
wherein the gasbag is partitioned into a first surface neighboring the arm of the testee and a second surface opposite to the first surface and far away from the arm of the testee while the cuff body is wound around an arm of a testee, a connection position where the tube is joined to the gasbag is on the second surface, the audio pickup probe is disposed on the second surface, and sound signals picked up by the audio pickup probe are used to determine a systolic pressure and a diastolic pressure.

2. The auscultatory sphygmomanometer system according to claim 1, wherein the audio pickup probe has a cone-shaped stethoscope disposed on a surface of the gasbag, and an audio sensor disposed on one end of the cone-shaped stethoscope having a smaller diameter.

3. The auscultatory sphygmomanometer system according to claim 2, wherein the audio sensor is a microelectromechanical system (MEMS) microphone; a response frequency of the MEMS microphone ranges from 10 Hz to 20 kHz; a sensitivity of the MEMS microphone is at least −45 dBV while a sound level calibrator outputs a sound signal having a frequency of 1 kHz and a sound pressure level of 94 dB.

4. The auscultatory sphygmomanometer system according to claim 1, wherein the pressure sensing and working module includes a gas valve connected with the tube, a gas pump connected with the tube, and a pressure sensor detecting a pressure inside the gasbag.

5. The auscultatory sphygmomanometer system according to claim 4, further comprising a signal processor connected with the pressure sensing and working module and the audio pickup probe, wherein the signal processor includes at least one first signal amplifier; a first multiple-band bandpass filter performing signal processing of the sound signals provided by the audio pickup probe; a second-order low pass filter performing signal processing of pressure signals provided by the pressure sensor to provide pressure values in a direct-current mode; and a second-order bandpass filter performing signal processing of the pressure signals provided by the pressure sensor to provide oscillometric pressure values in an alternating-current mode.

6. The auscultatory sphygmomanometer system according to claim 5, wherein the audio pickup probe includes a front-end signal processor connected with the audio sensor; the front-end signal processor includes a second signal amplifier, and a second multiple-band band pass filter succeeding to the second signal amplifier.

7. The auscultatory sphygmomanometer system according to claim 2, wherein the pressure sensing and working module includes a gas valve connected with the tube, a gas pump connected with the tube, and a pressure sensor detecting a pressure inside the gasbag.

8. The auscultatory sphygmomanometer system according to claim 7, further comprising a signal processor connected with the pressure sensing and working module and the audio pickup probe, wherein the signal processor includes at least one first signal amplifier; a first multiple-band bandpass filter performing signal processing of the sound signals provided by the audio pickup probe; a second-order low pass filter performing signal processing of pressure signals provided by the pressure sensor to provide pressure values in a direct-current mode; and a second-order bandpass filter performing signal processing of the pressure signals provided by the pressure sensor to provide oscillometric pressure values in an alternating-current mode.

9. The auscultatory sphygmomanometer system according to claim 8, wherein the audio pickup probe includes a front-end signal processor connected with the audio sensor; the front-end signal processor includes a second signal amplifier, and a second multiple-band band pass filter succeeding to the second signal amplifier.

10. The auscultatory sphygmomanometer system according to claim 3, wherein the pressure sensing and working module includes a gas valve connected with the tube, a gas pump connected with the tube, and a pressure sensor detecting a pressure inside the gasbag.

11. The auscultatory sphygmomanometer system according to claim 10, further comprising a signal processor connected with the pressure sensing and working module and the audio pickup probe, wherein the signal processor includes at least one first signal amplifier; a first multiple-band bandpass filter performing signal processing of the sound signals provided by the audio pickup probe; a second-order low pass filter performing signal processing of pressure signals provided by the pressure sensor to provide pressure values in a direct-current mode; and a second-order bandpass filter performing signal processing of the pressure signals provided by the pressure sensor to provide oscillometric pressure values in an alternating-current mode.

12. The auscultatory sphygmomanometer system according to claim 11, wherein the audio pickup probe includes a front-end signal processor connected with the audio sensor; the front-end signal processor includes a second signal amplifier, and a second multiple-band band pass filter succeeding to the second signal amplifier.

13. A method of using an auscultatory sphygmomanometer system to measure blood pressure, comprising
Step 1: providing the auscultatory sphygmomanometer system of claim 1; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

14. A method of using an auscultatory sphygmomanometer system to measure blood pressure, comprising
Step 1: providing the auscultatory sphygmomanometer system of claim 2; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

15. A method of using an auscultatory sphygmomanometer system to measure blood pressure, comprising
Step 1: providing the auscultatory sphygmomanometer system of claim 3; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

16. A method of using an auscultatory sphygmomanometer system to measure blood pressure, comprising
Step 1: providing the auscultatory sphygmomanometer system of claim 4; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

17. A method of using an auscultatory sphygmomanometer system to measure blood pressure, comprising
Step 1: providing the auscultatory sphygmomanometer system of claim 5; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

18. A method of using an auscultatory sphygmomanometer system to measure blood pressure, comprising
Step 1: providing the auscultatory sphygmomanometer system of claim 6; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

19. A method of using an auscultatory sphygmomanometer system to measure blood pressure, comprising
Step 1: providing the auscultatory sphygmomanometer system of claim 7; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

20. A method of using an auscultatory sphygmomanometer system to measure blood pressure, comprising
Step 1: providing the auscultatory sphygmomanometer system of claim 8; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

21. A method of using an auscultatory sphygmomanometer system to measure blood pressure, comprising
Step 1: providing the auscultatory sphygmomanometer system of claim 9; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

22. A method of using an auscultatory sphygmomanometer system to measure blood pressure, comprising
Step 1: providing the auscultatory sphygmomanometer system of claim 10; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

23. A method of using an auscultatory sphygmomanometer system to measure blood pressure, comprising
Step 1: providing the auscultatory sphygmomanometer system of claim 11; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.

24. A method of using an auscultatory sphygmomanometer system to measure blood pressure, comprising
Step 1: providing the auscultatory sphygmomanometer system of claim 12; and
Step 2: inflating the gasbag to a preset pressure under an action of the pressure sensing and working module, and then deflating the gasbag; the audio pickup probe using the second surface of the gasbag as the oscillation membrane, which generates a sound signal; acquiring a systolic pressure while the Korotkoff sound appears in the sound signal for the first time; acquiring a diastolic pressure while the Korotkoff sound appears in the sound signal for the last time.
